(19) United States
(12) Patent Application Publication
(10) Pub. No.: US 2026/0038299 A1
(43) Pub. Date: Feb. 5, 2026

(54) DETECTION DEVICE

(71) Applicant: Japan Display Inc., Tokyo (JP)

(72) Inventors: Atsunori OYAMA, Tokyo (JP); Gen KOIDE, Tokyo (JP)

(21) Appl. No.: 19/354,576

(22) Filed: Oct. 9, 2025

Related U.S. Application Data
(63) Continuation of application No. PCT/JP2024/012031, filed on Mar. 26, 2024.

(30) Foreign Application Priority Data
Apr. 14, 2023 (JP) 2023-066376

(51) Int. Cl. G06V 40/13; A61B 5/1172; H10K 39/32

(52) U.S. Cl. CPC G06V 40/1318; A61B 5/1172; H10K 39/32

(57) ABSTRACT

According to an aspect, a detection device includes: a substrate having a notch; a terminal part provided at one end in the first direction of the substrate; a first optical sensor provided between the notch and the terminal part; and a second optical sensor provided between the notch and another end of the substrate. The upper electrode of the first optical sensor is coupled to a first power supply electrode and coupled to the terminal part via a first wiring line coupled to the first power supply electrode. The upper electrode of the second optical sensor is coupled to a second power supply electrode and coupled to the terminal part via a second wiring line coupled to the second power supply electrode. The lower electrodes of the first and second optical sensors are coupled to the terminal part via third wiring lines.

### CROSS-REFERENCE TO RELATED APPLICATION

This application claims the benefit of priority from Japanese Patent Application No. 2023-066376 filed on April 14, 2023 and International Patent Application No. PCT/JP2024/012031 filed on March 26, 2024, the entire contents of which are incorporated herein by reference.

### BACKGROUND

### Technical Field

What is disclosed herein relates to a detection device.

### Description of the Related Art

Optical sensors capable of detecting fingerprint patterns and vein patterns are known (refer to, for example, Japanese Patent Application Laid-open Publication No. 2009-032005). Among such optical sensors, sensors are known each including a plurality of photodiodes in which an organic semiconductor material is used as an active layer. The organic semiconductor material is disposed between lower and upper electrodes, and signal lines are electrically coupled to the lower electrodes of the photodiodes to output detection signals to a detection circuit.

When a plurality of optical sensors are arranged and power is supplied from one common upper electrode to the optical sensors, a high sheet resistance of the upper electrode causes a difference in power supply capacity between elements in regions farther from the upper electrode (power source) and elements in regions closer thereto, resulting in differences in sensitivity among the optical sensors. The sheet resistance indicates the electrical resistance of a thin film, a film, or the like having a uniform small thickness. When the multiple optical sensors are supplied with power by one upper electrode, if a failure occurs in the optical sensors, all the optical sensors may become unusable.

For the foregoing reasons, there is a need for a detection device capable of reducing the effects of the differences in sensitivity and a failure of a plurality of optical sensors arranged in an array.

### SUMMARY

According to an aspect, a detection device includes: a substrate having opposite ends in a first direction with a notch therebetween; a terminal part provided at one end in the first direction of the substrate; a first optical sensor provided on the substrate between the notch and the terminal part; and a second optical sensor provided on the substrate between the notch and another end of the substrate. In each of the first optical sensor and the second optical sensor, a lower electrode, a lower buffer layer, an active layer, an upper buffer layer, an upper electrode, and a sealing film are stacked on the substrate in the order as listed. The upper electrode of the first optical sensor is coupled to a first power supply electrode and coupled to the terminal part via a first wiring line coupled to the first power supply electrode. The upper electrode of the second optical sensor is coupled to a second power supply electrode and coupled to the terminal part via a second wiring line coupled to the second power supply electrode. The lower electrodes of the first optical sensor and the second optical sensor are coupled to the terminal part via third wiring lines.

According to an aspect, a detection device includes: a substrate extending in a first direction; a terminal part provided at one end in the first direction of the substrate; a plurality of optical sensors arranged along the first direction on the substrate. In each of the optical sensors, a lower electrode, a lower buffer layer, an active layer, an upper buffer layer, an upper electrode, and a sealing film are stacked on the substrate in the order as listed. Each of the upper electrodes of the optical sensors is coupled to a power supply electrode and coupled to the terminal part via a first wiring line coupled to the power supply electrode. Each of the lower electrodes of the optical sensors is coupled to the terminal part via a third wiring line.

### BRIEF DESCRIPTION OF THE DRAWINGS

FIG. 1 is a schematic view illustrating an exemplary external appearance when a state of a finger accommodated inside a detection device according to a first embodiment is viewed from a lateral side of a housing;

FIG. 2 is a schematic sectional view taken along section A-A illustrated in FIG. 1;

FIG. 3 is a development view illustrating an exemplary development of optical sensors of the detection device illustrated in FIG. 1;

FIG. 4 is a schematic top view illustrating an exemplary configuration of a substrate illustrated in FIG. 3;

FIG. 5 is a schematic sectional view illustrating an exemplary multilayer configuration of an optical sensor taken along section B-B illustrated in FIG. 4;

FIG. 6 is a schematic sectional view illustrating an exemplary multilayer configuration of an optical sensor taken along section C-C illustrated in FIG. 4;

FIG. 7 is a schematic top view illustrating an exemplary configuration of a reference example substrate according to the first embodiment;

FIG. 8 is a schematic top view illustrating an exemplary configuration of the substrate of the detection device according to a second embodiment;

FIG. 9 is a schematic top view illustrating an exemplary configuration of a substrate of the detection device according to a third embodiment;

FIG. 10 is a schematic sectional view illustrating an exemplary multilayer configuration of the optical sensor taken along section D-D illustrated in FIG. 9;

FIG. 11 is a schematic top view illustrating an exemplary configuration of a reference example substrate according to the third embodiment; and

FIG. 12 is a schematic top view illustrating an exemplary configuration of the substrate of the detection device according to a fourth embodiment.

### DETAILED DESCRIPTION

The following describes modes (embodiments) for carrying out the disclosure in detail with reference to the drawings. The present disclosure is not limited to the description of the embodiments given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the present specification and the drawings, and detailed description thereof may not be repeated where appropriate.

In the present specification and claims, in expressing an aspect of disposing another structure on or above a certain structure, a case of simply expressing "on" includes both a case of disposing the other structure immediately on the certain structure so as to contact the certain structure and a case of disposing the other structure above the certain structure with still another structure interposed therebetween, unless otherwise specified.

### First Embodiment

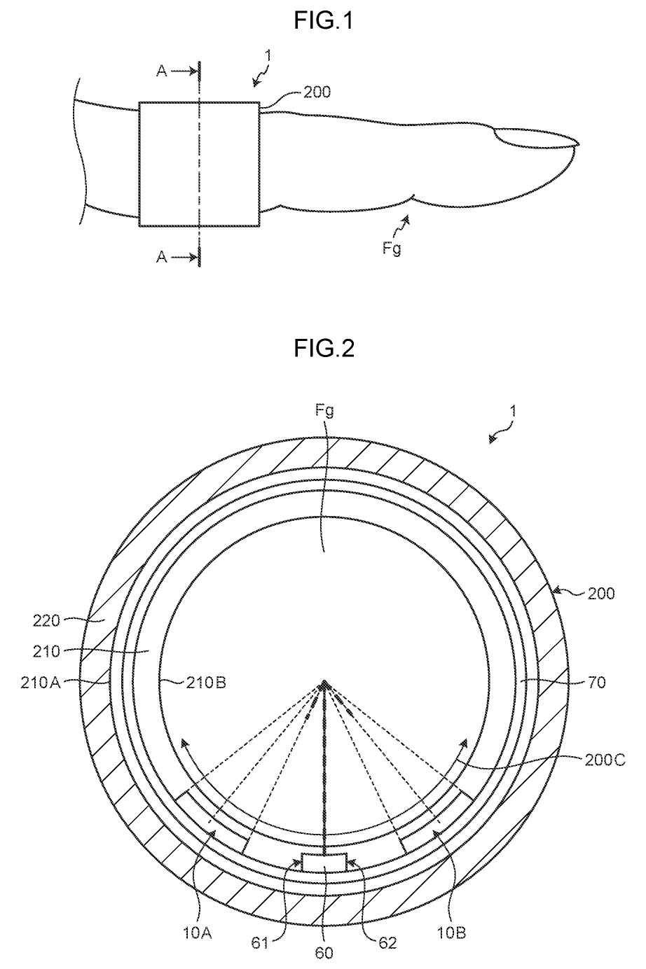

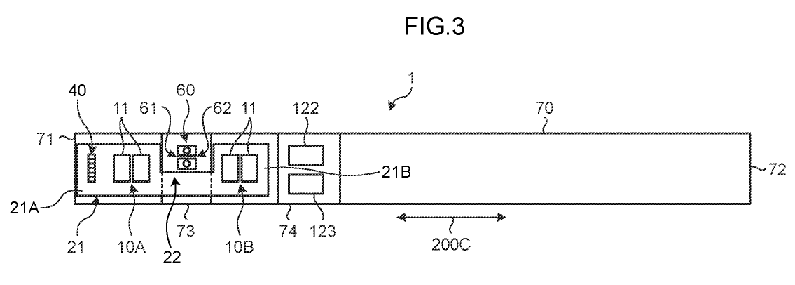

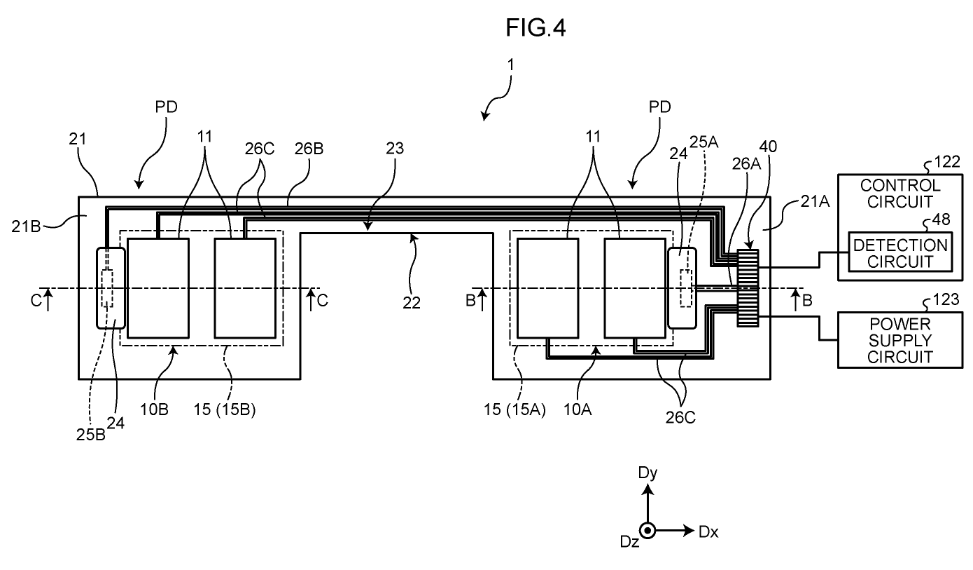

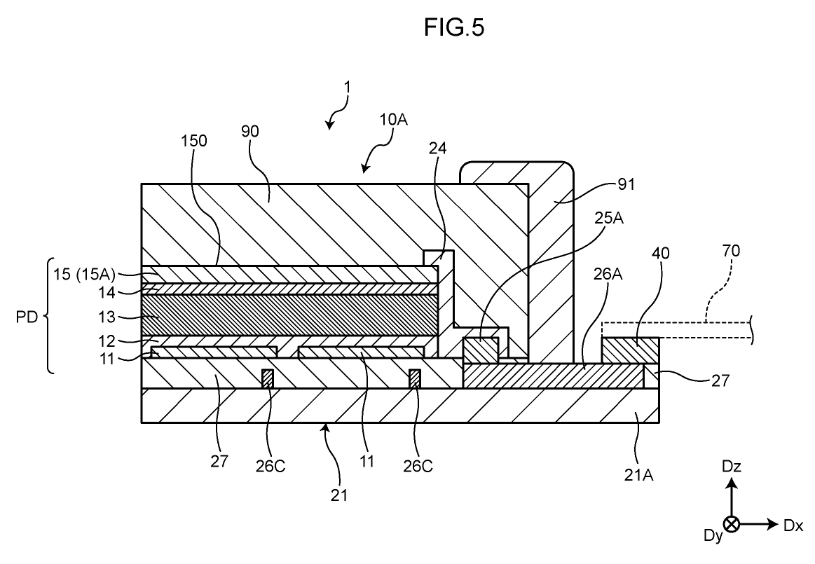

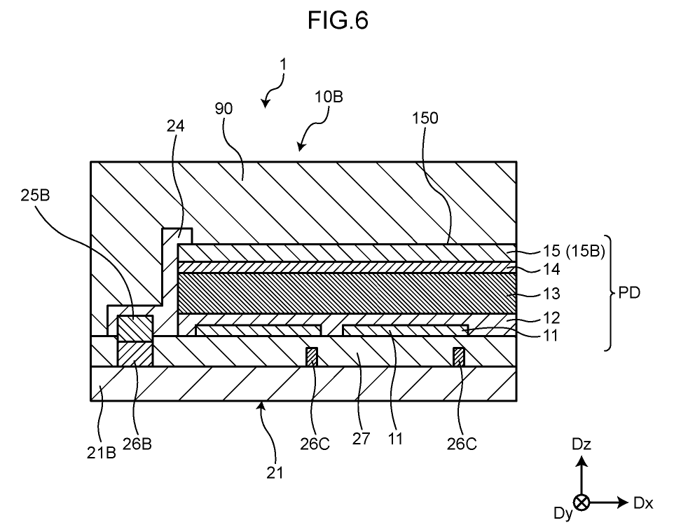

FIG. 1 is a schematic view illustrating an exemplary external appearance when a state of a finger accommodated inside a detection device according to a first embodiment is viewed from a lateral side of a housing. FIG. 2 is a schematic sectional view taken along section A-A illustrated in FIG. 1. FIG. 3 is a development view illustrating an exemplary development of optical sensors of the detection device illustrated in FIG. 1. FIG. 4 is a schematic top view illustrating an exemplary configuration of a substrate illustrated in FIG. 3. FIG. 5 is a schematic sectional view illustrating an exemplary multilayer configuration of an optical sensor taken along section B-B illustrated in FIG. 4. FIG. 6 is a schematic sectional view illustrating an exemplary multilayer configuration of an optical sensor taken along section C-C illustrated in FIG. 4.

A detection device 1 illustrated in FIG. 1 is a finger ring-shaped device that can be worn on and removed from a human body and is worn on a finger Fg of the human body. Examples of the finger Fg include a thumb, an index finger, a middle finger, a ring finger, and a little finger. The human body is a person to be authenticated whose identity is to be verified by the detection device 1. The detection device 1 can detect biometric information on a living body from the finger Fg wearing the detection device 1. The finger Fg is an example of a measurement target. The measurement target is the living body or a part of the living body, and is an object to be measured. The detection device 1 is formed as a finger ring or a wristband so as to be easily carried by a user. In the following description, the detection device 1 is assumed to be used as a finger ring.

As illustrated in FIG. 2, the detection device 1 includes a housing 200, a light source 60, a first optical sensor 10A, a second optical sensor 10B, and a flexible printed circuit board 70. The detection device 1 is a device that includes a battery (not illustrated) in the housing 200 and is operated by power from the battery.

The housing 200 is formed in a ring shape (annular shape) that can be worn on the finger Fg, and is a wearable member to be worn on the living body. In the example illustrated in FIG. 2, the housing 200 includes a first housing 210 and a second housing 220. The first housing 210 is integrated with the second housing 220 to form the housing 200 into a ring shape. The first housing 210 is a member that makes contact with the human body wearing the housing 200. The first housing 210 accommodates therein the light source 60, the first optical sensor 10A, the second optical sensor 10B, and so forth. The first housing 210 is formed into a ring shape using a housing material such as a light-transmitting synthetic resin or silicon. The second housing 220 has a surface of the housing 200 that covers an outer peripheral surface 210A of the first housing 210. The second housing 220 is formed into a ring shape using a member of, for example, a metal or a non-light-transmitting synthetic resin. The first housing 210 of the housing 200 accommodates the flexible printed circuit board 70 on which the light source 60, the first optical sensor 10A, the second optical sensor 10B, and so forth are mounted. The flexible printed circuit board 70 is accommodated in the housing 200, for example, by forming the housing 200 by filling the periphery of the flexible printed circuit board 70 formed into a ring shape with a filling member in a mold.

As illustrated in FIG. 3, the flexible printed circuit board 70 is formed into a deformable band shape, and is formed into the ring shape by coupling one end 71 to the other end 72. The flexible printed circuit board 70 has a first mounting area 73 and a second mounting area 74. The first mounting area 73 is an area where the light source 60 and so forth are mounted. The second mounting area 74 is an area where a control circuit 122, a power supply circuit 123, and so forth are mounted. A substrate 21 is mounted on the flexible printed circuit board 70 so as to straddle the vicinity of the light source 60 in the first mounting area 73. The substrate 21 is a sensor substrate on which the first and the second optical sensors 10A and 10B are fabricated. The flexible printed circuit board 70 electrically couples the light source 60, the first optical sensor 10A, the second optical sensor 10B, and so forth to the control circuit 122.

In the present embodiment, the first and the second optical sensors 10A and 10B are provided so as to interpose the light source 60 therebetween in a circumferential direction 200C. That is, in the detection device 1, the first optical sensor 10A, the light source 60, and the second optical sensor 10B are arranged in this order in the circumferential direction 200C. The first and the second optical sensors 10A and 10B are arranged so as to interpose the light source 60 therebetween in the circumferential direction 200C. Thereby, light emitted by the light source 60 can be detected over a wide area of the housing 200.

The detection device 1 includes the substrate 21, and further a terminal part 40. The substrate 21 is an insulating substrate and is formed, for example, of a film-like resin or the like and into a band shape. The substrate 21 is a deformable substrate on which the first and the second optical sensors 10A and 10B are fabricated. The sensor substrate 21 is mounted on the flexible printed circuit board 70, whereby the first and the second optical sensors 10A and 10B are positioned on opposite sides of the light source 60 in the circumferential direction 200C of the housing 200. The substrate 21 has a notch 22 between both opposite ends of the substrate 21 in the circumferential direction 200C of the housing 200, that is, between the two longitudinal ends of the substrate 21. On the substrate 21, the first optical sensor 10A is fabricated on one end 21A side and the second optical sensor 10B is fabricated on the other end 21B side with the notch 22 interposed therebetween. The terminal part 40 is provided at the one end 21A in the longitudinal direction of the substrate 21. The terminal part 40 supplies power from the power supply circuit 123 to the first and the second optical sensors 10A and 10B.

In the present embodiment, as illustrated in FIG. 2, the flexible printed circuit board 70 is accommodated in the housing 200 such that a surface provided with the first optical sensor 10A, the second optical sensor 10B, and the light source 60 faces an inner peripheral surface 200B of the housing 200. When the flexible printed circuit board 70 has a light-transmitting property, the first optical sensor 10A, the second optical sensor 10B, and the light source 60 may be mounted on the back surface opposite the front surface. In this case, the light source 60 only needs to be disposed such that light is emitted toward the flexible printed circuit board 70 and light transmitted through the flexible printed circuit board 70 is emitted toward outside the housing 200.

As illustrated in FIG. 2, the light source 60 is provided in the first housing 210 of the housing 200 and is configured to be capable of emitting light toward the finger Fg wearing the housing 200. For example, inorganic light-emitting diodes (LEDs) or organic electroluminescent (EL) diodes (organic light-emitting diodes (OLEDs)) are used as the light source 60. The light source 60 emits light having predetermined wavelengths. In the present embodiment, the light source 60 includes a plurality of light sources so as to be capable of emitting near-infrared light, red light, and green light.

The light emitted from the light source 60 is reflected by a surface of an object to be detected, such as the finger Fg, and enters the first and the second optical sensors 10A and 10B. Thereby, the detection device 1 can detect a fingerprint by detecting a shape of asperities on the surface of the finger Fg or the like. Alternatively, the light emitted from the light source 60 may be reflected in the finger Fg or the like, or transmitted through the finger Fg or the like and enter the first and the second optical sensors 10A and 10B. Thereby, the detection device 1 can detect the information on the living body in the finger Fg or the like. Examples of the information on the living body include, but are not limited to, pulse waves, pulsation, and a vascular image of the finger or a palm. That is, the detection device 1 may be configured as a fingerprint detection device that detects the fingerprint or a vein detection device that detects a vascular pattern of, for example, veins.

Each of the first and the second optical sensors 10A and 10B detects the light emitted by the light source 60 and reflected by the finger Fg or the like, light directly incident on the optical sensor, and other light. The first and the second optical sensors 10A and 10B are each an organic photodiode (OPD). The first optical sensor 10A is provided in the housing 200 so as to be adjacent to one end 61 of the light source 60 in the circumferential direction 200C of the housing 200. The second optical sensor 10B is provided in the housing 200 so as to be adjacent to another end 62 of the light source 60 in the circumferential direction 200C of the housing 200.

As illustrated in FIG. 3, the first and the second optical sensors 10A and 10B each have a photodiode PD (refer to FIG. 4) that is an organic photodiode. Each of the first and the second optical sensors 10A and 10B has a configuration with two lower electrodes 11 arranged along the circumferential direction 200C. The first and the second optical sensors 10A and 10B are fabricated on one substrate 21 and are electrically coupled to the flexible printed circuit board 70 via the substrate 21. The substrate 21 has the notch 22 between the first and the second optical sensors 10A and 10B in the circumferential direction 200C of the housing 200. The notch 22 will be described later.

In the following description, a first direction Dx is one direction in a plane parallel to the substrate 21 and is the same direction as the circumferential direction 200C. A second direction Dy is one direction in the plane parallel to the substrate 21 and is a direction orthogonal to the first direction Dx. The second direction Dy may non-orthogonally intersect the first direction Dx. A third direction Dz is a direction orthogonal to the first direction Dx and the second direction Dy. The third direction Dz is a direction normal to the substrate 21. The term "plan view" refers to a positional relation when viewed along a direction orthogonal to the substrate 21.

As illustrated in FIG. 4, the first optical sensor 10A has a configuration in which one upper electrode 15A is stacked on the two lower electrodes 11 arranged in the first direction Dx so as to cover the lower electrodes 11. The second optical sensor 10B has a configuration in which one upper electrode 15B is stacked on the two lower electrodes 11 arranged in the first direction Dx so as to cover the lower electrodes 11. An upper electrode 15 includes the upper electrode 15A of the first optical sensor 10A and the upper electrode 15B of the second optical sensor 10B. Each of the upper electrode 15A and the upper electrode 15B covers the two lower electrodes 11 in plan view. The upper electrode 15A and the upper electrode 15B have each a rectangular surface shape and are an independent electrodes that are not electrically coupled to each other.

The substrate 21 includes a first power supply electrode 25A and a second power supply electrode 25B that extend along the second direction Dy. The first power supply electrode 25A is provided between the one end 21A side in the first direction Dx of the substrate 21 and the first optical sensor 10A. The second power supply electrode 25B is provided between the other end 21B side in the first direction Dx of the substrate 21 and the second optical sensor 10B. The first power supply electrode 25A is electrically coupled to the terminal part 40 of the substrate 21 via a first wiring line 26A and is supplied with a power supply signal from the power supply circuit 123 (refer to FIG. 3) via the terminal part 40. The second power supply electrode 25B is electrically coupled to the terminal part 40 of the substrate 21 via a second wiring line 26B and is supplied with a power supply signal from the power supply circuit 123 via the terminal part 40.

The upper electrode 15A of the first optical sensor 10A is coupled to the first power supply electrode 25A via a conductor 24 and electrically coupled to the terminal part 40 via the first wiring line 26A coupled to the first power supply electrode 25A. The upper electrode 15B of the second optical sensor 10B is coupled to the second power supply electrode 25B via a conductor 24 and electrically coupled to the terminal part 40 via the second wiring line 26B coupled to the second power supply electrode 25B. With this configuration, the upper electrodes 15A and 15B are supplied with power from independent power systems of the first and the second power supply electrodes 25A and 25B, respectively. Each of the conductors 24 is formed of a conductive material and covers the entire surface of the first power supply electrode 25A or the second power supply electrode 25B, thus electrically coupling the first power supply electrode 25A to the upper electrode 15A or the second power supply electrode 25B to the upper electrode 15B. The upper electrode 15A and the upper electrode 15B may be directly coupled to the first power supply electrode 25A and the second power supply electrode 25B without the conductors 24 interposed therebetween.

Each of the lower electrodes 11 of the first and the second optical sensors 10A and 10B is coupled to the terminal part 40 via third wiring lines 26C. The third wiring lines 26C of the substrate 21 are coupled to a detection circuit 48 included in the control circuit 122 via the terminal part 40 and signal lines of the flexible printed circuit board 70. In other words, the detection circuit 48 is electrically coupled to the lower electrodes 11 of the first and the second optical sensors 10A and 10B via the signal lines. The detection circuit 48 may be formed as a circuit separate from the control circuit 122.

The first and the second power supply electrodes 25A and 25B are supplied with the power supply signals from the power supply circuit 123 via the terminal part 40, and supplies the power supply signals to the upper electrodes 15A and 15B. In the example with reference to FIG. 4, the first and the second power supply electrodes 25A and 25B are formed in a substantially rectangular shape extending in the second direction Dy in plan view and have the same area (size).

As illustrated in FIG. 5, the first optical sensor 10A includes the substrate 21 and the photodiode PD. In the present embodiment, the first optical sensor 10A further includes the third wiring lines 26C, an insulating layer 27, and a sealing film 90.

The third wiring lines 26C are provided on the upper surface of the substrate 21. The third wiring lines 26C are formed, for example, of metal wiring lines, and are formed of a material having better conductivity than the lower electrodes 11 of the photodiode PD. The third wiring lines 26C are provided in a layer between the substrate 21 and the photodiode PD in the third direction Dz. The third wiring lines 26C are electrically coupled to the terminal part 40 on the substrate 21 (refer to FIG. 4). The third wiring lines 26C may be formed, for example, in the same layer as the lower electrodes 11, and/or formed of a metal. The insulating layer 27 is provided on the substrate 21 so as to cover the third wiring lines 26C. The insulating layer 27 may be an inorganic insulating film or an organic insulating film.

The photodiode PD is provided as a sensor element on the insulating layer 27. The photodiode PD includes the lower electrodes 11, a lower buffer layer 12, an active layer 13, an upper buffer layer 14, and the upper electrode 15 (15A). In the photodiode PD, the lower electrodes 11, the lower buffer layer 12 (hole transport layer), the active layer 13, the upper buffer layer 14 (electron transport layer), and the upper electrode 15 are stacked in this order in the third direction Dz orthogonal to the substrate 21.

Each of the lower electrodes 11 is an anode electrode of the photodiode PD, and is formed of a light-transmitting conductive material such as indium tin oxide (ITO), for example. The active layer 13 changes in characteristics (such as voltage-current characteristics and resistance value) depending on light emitted thereto. An organic material is used as a material of the active layer 13. Specifically, the active layer 13 has a bulk heterostructure containing a mixture of a p-type organic semiconductor and an n-type fullerene derivative ((6,6)-phenyl-C₆₁-butyric acid methyl ester (PCBM)) that is an n-type organic semiconductor. As the active layer 13, low-molecular-weight organic materials can be used including, for example, fullerene (C₆₀), phenyl-C₆₁-butyric acid methyl ester (PCBM), copper phthalocyanine (CuPc), fluorinated copper phthalocyanine (F₁₆CuPc), 5,6,11,12-tetraphenyltetracene (rubrene), and perylene diimide (PDI) (derivative of perylene).

The active layer 13 can be formed by a vapor deposition process (dry process) using any of the low-molecular-weight organic materials listed above. In this case, the active layer 13 may be, for example, a multilayered film of CuPc and F₁₆CuPc, or a multilayered film of rubrene and C₆₀. The active layer 13 can also be formed by a coating process (wet process). In this case, the active layer 13 is made using a material obtained by combining any of the above-listed low-molecular-weight organic materials with a high-molecular-weight organic material. As the high-molecular-weight organic material, for example, poly(3-hexylthiophene) (P3HT) and F8-alt-benzothiadiazole (F8BT) can be used. The active layer 13 can be a film made of a mixture of P3HT and PCBM, or a film made of a mixture of F8BT and PDI.

The lower buffer layer 12 is a hole transport layer. The upper buffer layer 14 is an electron transport layer. The lower buffer layer 12 and the upper buffer layer 14 are provided to facilitate holes and electrons generated in the active layer 13 to reach the lower electrodes 11 or the upper electrode 15. The lower buffer layer 12 (hole transport layer) is in direct contact with the tops of the lower electrodes 11 and is also provided in an area between the adjacent lower electrodes 11. The active layer 13 is in direct contact with the top of the lower buffer layer 12. The material of the hole transport layer is a metal oxide layer. Tungsten oxide (WO₃), molybdenum oxide, or the like is used as the metal oxide layer.

The upper buffer layer 14 (electron transport layer) is in direct contact with the top of the active layer 13, and the upper electrode 15 is in direct contact with the top of the upper buffer layer 14. Polyethylenimine ethoxylated (PEIE) is used as a material of the electron transport layer.

The materials and the manufacturing methods of the lower buffer layer 12, the active layer 13, and the upper buffer layer 14 are merely exemplary, and other materials and manufacturing methods may be used. For example, each of the lower buffer layer 12 and the upper buffer layer 14 is not limited to a single-layer film, and may be formed as a multilayered film that includes an electron blocking layer and a hole blocking layer.

The upper electrode 15 is provided on the upper buffer layer 14. The upper electrode 15 is a cathode electrode of the photodiode PD and is continuously formed over the entire first and second optical sensors 10A and 10B. In other words, the upper electrode 15 is continuously provided on the photodiodes PD. The upper electrode 15 faces the lower electrodes 11 with the lower buffer layer 12, the active layer 13, and the upper buffer layer 14 interposed therebetween. The upper electrode 15 is formed, for example, of a light-transmitting conductive material such as ITO or indium zinc oxide (IZO). A portion of an end of an upper surface 150 of the upper electrode 15 is electrically coupled to the conductor 24. The conductor 24 is electrically coupled to the first power supply electrode 25A and supplies the power supply signal from the first power supply electrode 25A to the upper electrode 15. In the first optical sensor 10A, the photodiode PD is well sealed by providing the sealing film 90 on the upper electrode 15, the conductor 24, and so forth. The upper electrode 15 may be a multilayered film of a plurality of light-transmitting conductive materials.

The sealing film 90 is provided on the upper electrode 15. An inorganic insulating film, such as a silicon nitride film or an aluminum oxide film, or a resin film, such as an acrylic film, is used as the sealing film 90. The sealing film 90 is not limited to a single layer, but may be a multilayered film having two or more layers obtained by combining the inorganic film with the resin film mentioned above. The sealing film 90 well seals the photodiode PD, and thus can reduce moisture entering the photodiode PD from the upper surface side thereof. In the present embodiment, the first optical sensor 10A is configured to protect the terminal part 40, the substrate 21, and so forth by covering from the sealing film 90 to a portion of the terminal part 40 with a resin 91.

As illustrated in FIG. 6, the second optical sensor 10B includes the two lower electrodes 11 of the second optical sensor 10B in areas of the substrate 21 different from those of the lower electrodes 11 of the first optical sensor 10A. The lower electrodes 11 are covered with the lower buffer layer 12, the active layer 13, the upper buffer layer 14, and the upper electrode 15 (15B). In the present embodiment, the second optical sensor 10B includes the substrate 21, the photodiode PD, the third wiring lines 26C, and the insulating layer 27. The photodiode PD, the third wiring lines 26C, and the insulating layer 27 of the second optical sensor 10B have the same configurations as those of the photodiode PD, the third wiring lines 26C, and the insulating layer 27 of the first optical sensor 10A described above. That is, the photodiode PD of the second optical sensor 10B includes the lower electrodes 11, the lower buffer layer 12, the active layer 13, the upper buffer layer 14, and the upper electrode 15 (15B). In the present embodiment, the first and the second optical sensors 10A and 10B are organic photodiodes.

In the second optical sensor 10B, the portion of the end of an upper surface 150 of the upper electrode 15 is electrically coupled to the conductor 24, and the conductor 24 is electrically coupled to the second power supply electrode 25B. The second optical sensor 10B supplies a power supply signal from the second power supply electrode 25B to the upper electrode 15. In the second optical sensor 10B, the photodiode PD is well sealed by providing the sealing film 90 on the upper electrode 15, the conductor 24, and so forth.

As illustrated in FIG. 4, the substrate 21 has an area of the first optical sensor 10A and an area of the second optical sensor 10B, and is integrally formed into one common substrate. The substrate 21 has the notch 22 formed between the area of the first optical sensor 10A and the area of the second optical sensor 10B in the first direction Dx. The substrate 21 includes the notch 22 and a joint 23, the notch 22 is provided between the first and the second optical sensors 10A and 10B, and the joint 23 is in contact with the notch 22 and lies between the first and the second optical sensors 10A and 10B.

The notch 22 is formed to have a length longer than the length of the light source 60 in the first direction Dx. The notch 22 is formed to have a length longer than the length of the light source 60 and shorter than the length (width) of the substrate 21 in the second direction Dy. The substrate 21 is integrally formed by connecting together the areas of the first and the second optical sensors 10A and 10B via the joint 23 beside the notch 22. The notch 22 is formed in a shape that allows the light source 60 to be located therein. In the present embodiment, the notch 22 is formed into a substantially rectangular shape in plan view, but may have a semicircular, triangular, polygonal, or other shape, for example. The joint 23 is provided with the second wiring line 26B and the third wiring lines 26C.

The terminal part 40 is electrically coupled to the flexible printed circuit board 70 (refer to FIG. 5). The terminal part 40 is a device for electrically coupling the first and the second optical sensors 10A and 10B on the substrate 21 to the control circuit 122 and the power supply circuit 123 on the flexible printed circuit board 70. The terminal part 40 is fabricated on the substrate 21 and electrically coupled to the first wiring line 26A, the second wiring line 26B, the third wiring lines 26C, and so forth on the substrate 21. The first wiring line 26A, the second wiring line 26B, and the third wiring lines 26C are metal lines in the same layer of the substrate 21. The terminal part 40 supplies the power supply signals (electric power) from the power supply circuit 123 to the first optical sensor 10A via the first wiring line 26A. The terminal part 40 supplies the power supply signals (electric power) from the power supply circuit 123 to the second optical sensor 10B via the second wiring line 26B. The terminal part 40 includes a plurality of terminals, thus, being able be coupled to a plurality of wiring lines.

The control circuit 122 is a circuit that controls detection operations by supplying control signals to the photodiodes PD. Each of the photodiodes PD outputs an electrical signal in response to light emitted thereto as a detection signal Vdet to the detection circuit 48. In the present embodiment, the detection signals Vdet of the photodiodes PD are sequentially output to the detection circuit 48 in a time-divisional manner. In other words, the signal lines SL are sequentially electrically coupled to the detection circuit 48 in a time-division manner. Thereby, the detection device 1 detects information on the object to be detected based on the detection signals Vdet from the photodiodes PD.

The exemplary configuration of the detection device 1 according to the present embodiment has been described above. The configuration described above using FIGS. 1 to 6 is merely an example, and the configuration of the detection device 1 according to the present embodiment is not limited to the example. The configuration of the detection device 1 according to the present embodiment can be flexibly modified depending on requirements or operations.

### Example of Detection by Detection Device Worn on Finger

The following describes an example of detection by the detection device 1 worn on the finger Fg. In the example illustrated in FIG. 2, the detection device 1 is in a state where an inner peripheral surface 210B of the first housing 210 of the housing 200 is in contact with or in proximity to the finger Fg. As illustrated in FIG. 4, the detection device 1 supplies a power supply signal from the power supply circuit 123 to the upper electrode 15A via the first power supply electrode 25A and a power supply signal from the power supply circuit 123 to the upper electrode 15B via the second power supply electrode 25B. In the detection device 1, the light source 60 is turned on to emit the light toward the finger Fg. The light source 60 emits the light toward one side and the other side in the circumferential direction 200C. In the detection device 1, the first and the second optical sensors 10A and 10B receive the light reflected by the finger Fg or the like. The detection device 1 detects the information on the living body of the finger Fg based on an amount of light detected by each of the two photodiodes PD of the first and the second optical sensors 10A and 10B.

Thus, since the first optical sensor 10A is located adjacent to the one end 61 of the light source 60 and the second optical sensor 10B is located adjacent to the other end 62 of the light source in the circumferential direction 200C of the housing 200, the detection device 1 can detect the light reflected by the finger Fg over a wide area. The detection device 1 can operate the two lower electrodes 11 by supplying the power from the first power supply electrode 25A to the upper electrode 15A of the first optical sensor 10A, and operate the two lower electrodes 11 by supplying the power from the second power supply electrode 25B to the upper electrode 15B of the second optical sensor 10B. Thus, even when the two optical sensors of the first and second optical sensors 10A and 10B are arranged in the circumferential direction 200C with the light source 60 interposed therebetween, the detection device 1 can supply the power from the first and the second power supply electrodes 25A and 25B to the upper electrodes 15A and 15B. Thus, the resistance of paths between the upper electrodes 15A and 15B and the terminal part 40 is made lower than when using one common upper electrode, whereby the detection device 1 can reduce the difference in power supply capacity between the first and second optical sensors 10A and 10B, and reduce a difference in sensitivity between the first and second optical sensors 10A and 10B. In addition, the multiple power supply electrodes are employed in the detection device 1, which can reduce the likelihood that both the first and second optical sensors 10A and 10B become unusable even if a failure occurs among the multiple optical sensors. As a result, the detection device 1 can detect the information on the living body of the finger Fg over a long time, even if the housing 200 is small and difficult to repair.

In the detection device 1, the first and the second wiring lines 26A and 26B are metal lines in the same layer as the third wiring lines 26C coupled to the lower electrodes 11 on the substrate 21. Therefore, the detection device 1 can arrange the first and the second power supply electrodes 25A and 25B on the substrate 21 without complicating the configuration of the substrate 21.

In the detection device 1, the first power supply electrode 25A is provided between the one end 21A side of the substrate 21 and the first optical sensor 10A in the first direction Dx, and the second power supply electrode 25B is provided between the other end 21B side of the substrate 21 and the second optical sensor 10B in the first direction Dx. This configuration allows the detection device 1 to be provided with the first and the second optical sensors 10A and 10B on the substrate 21 near the notch 22, so that the first and the second optical sensors 10A and 10B can be brought closer to each other with the notch 22 interposed therebetween.

The detection device 1 includes the light source 60 located in the notch 22 of the substrate 21. As a result, in the detection device 1, the first and the second optical sensors 10A and 10B can be arranged near the notch 22 of the substrate 21. By being closer to the light source 60, the first and the second optical sensors 10A and 10B can be improved in sensitivity.

### Contrast with Reference Example Substrate According to First Embodiment

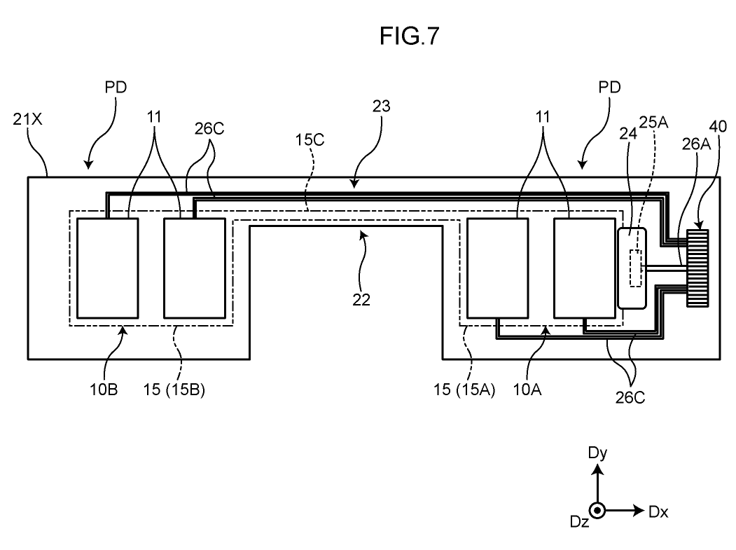

FIG. 7 is a schematic top view illustrating an exemplary configuration of a reference example substrate according to the first embodiment. On a reference example substrate 21X illustrated in FIG. 7, the upper electrodes 15A and 15B are integrally formed via an electrode connector 15C of the joint 23. The lower buffer layer 12, the active layer 13, the upper buffer layer 14, and the electrode connector 15C for the upper electrodes 15 are arranged at the joint 23. The electrode connector 15C and the upper electrodes 15 of the first and the second optical sensors 10A and 10B are integrally formed. In the reference example substrate 21X, power is supplied from one first power supply electrode 25A to the first and the second optical sensors 10A and 10B. Therefore, since the power is supplied to the first and the second optical sensors 10A and 10B through one common upper electrode 15 on the reference example substrate 21X, the resistance value of a path to the upper electrode 15 is higher as the distance from the first power supply electrode 25A becomes larger. Thus, a difference in power supply capacity occurs between an element farther from the first power supply electrode 25A and an element closer to the first power supply electrode 25A, resulting in a difference in sensitivity between the first and the second optical sensors 10A and 10B.

In contrast, the detection device 1 according to the first embodiment is configured to supply the power from the first power supply electrode 25A to the upper electrode 15A of the first optical sensor 10A and supply the power from the second power supply electrode 25B to the upper electrode 15B of the second optical sensor 10B. This configuration inhibits an increase in the resistance values of the paths from the terminal part 40 to the upper electrodes 15A and 15B in the detection device 1, so that the difference in sensitivity between the first and the second optical sensors 10A and 10B can be reduced. In addition, the multiple power supply electrodes are employed in the detection device 1 according to the first embodiment, which can reduce the likelihood that all the optical sensors become unusable even if a failure occurs in a sensor element of the first optical sensor 10A or the second optical sensor 10B.

### Second Embodiment

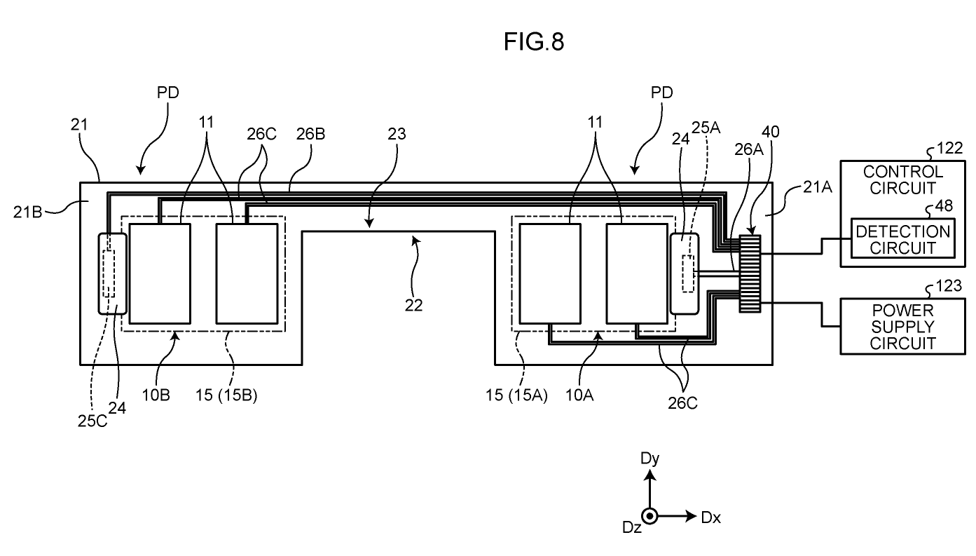

FIG. 8 is a schematic top view illustrating an exemplary configuration of the substrate 21 of the detection device 1 according to a second embodiment. In the second embodiment, the detection device 1 includes the substrate 21, the terminal part 40, the first optical sensor 10A, and the second optical sensor 10B described above. The substrate 21 includes the first power supply electrode 25A and a second power supply electrode 25C that extend along the second direction Dy. The second power supply electrode 25C is formed to have the same length in the first direction Dx as the second power supply electrode 25B described above, and to be longer in length in the second direction Dy intersecting the first direction Dx, and larger in electrode area than the first power supply electrode 25A. The second power supply electrode 25C is electrically coupled to the terminal part 40 of the substrate 21 via the second wiring line 26B and is supplied with the power supply signal from the power supply circuit 123 via the terminal part 40. The upper electrode 15B of the second optical sensor 10B is coupled to the second power supply electrode 25C and electrically coupled to the terminal part 40 via the second wiring line 26B coupled to the second power supply electrode 25B. That is, the upper electrode 15B of the second optical sensor 10B has a larger contact area with the second power supply electrode 25C than when using the first power supply electrode 25A. The upper electrodes 15A and 15B are supplied with power from the independent systems of the first and the second power supply electrodes 25A and 25C, respectively.

Thus, in the detection device 1, it is possible, by reducing the resistance of the second power supply electrode 25C, to inhibit an increase in the resistance of a path from the terminal part 40 to the upper electrode 15B, even if the distance from the terminal part 40 to the second power supply electrode 25C is large on the substrate 21. As a result, in the detection device 1, the difference between the resistance of the path from the terminal part 40 to the upper electrode 15A and the resistance of the path from the terminal part 40 to the upper electrode 15B can be reduced. Consequently, in the detection device 1, the difference in sensor sensitivity between the first optical sensor 10A closer to the terminal part 40 and the second optical sensor 10B that is farther than the first optical sensor 10A from the terminal part 40 can be reduced, even with an increase in size of the substrate 21.

In the example illustrated in the second embodiment, the second power supply electrode 25C is formed with a longer length in the second direction Dy than the first power supply electrode 25A, but the length in the first direction Dx may be longer. The second power supply electrode 25C may have the same length and surface area as the first power supply electrode 25A, and may have a different thickness therefrom.

### Third Embodiment

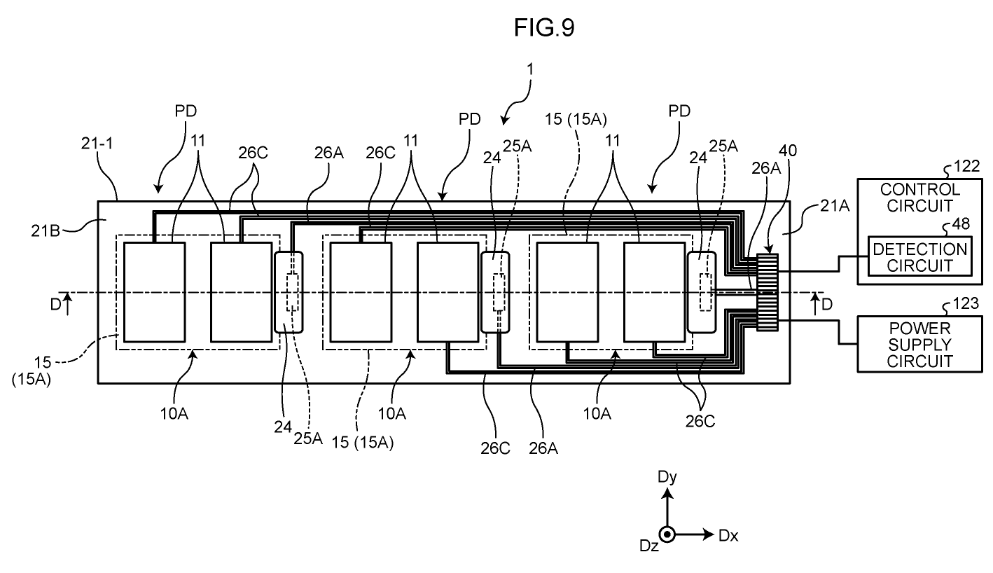

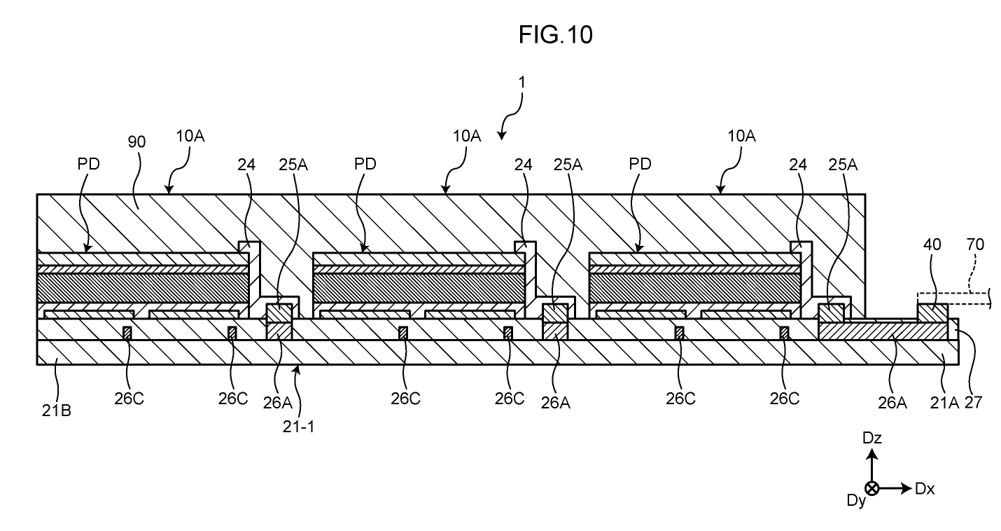

FIG. 9 is a schematic top view illustrating an exemplary configuration of a substrate of the detection device according to a third embodiment. FIG. 10 is a schematic sectional view illustrating an exemplary multilayer configuration of the optical sensor taken along section D-D illustrated in FIG. 9. As illustrated in FIGS. 9 and 10, the detection device 1 includes the terminal part 40 and the first optical sensor 10A described above, and a substrate 21-1.

The substrate 21-1 is formed into a band shape extending along the first direction Dx and does not have the notch 22 described above. The substrate 21-1 is a deformable substrate on which three first optical sensors 10A are fabricated along the first direction Dx. In the present embodiment, the case in which the three first optical sensors 10A are fabricated on the substrate 21-1 will be described, but the number of the first optical sensors 10A is not limited to this case.

The substrate 21-1 is mounted on the flexible printed circuit board 70, and the three first optical sensors 10A are arranged at predetermined intervals in the circumferential direction 200C of the housing 200. Example of the predetermined intervals include, but are not limited to, intervals at which the first power supply electrodes 25A can be arranged. In the detection device 1, for example, the light source 60 may be located on the other end 21B side of the substrate 21-1, or the substrate 21-1 and the light source 60 may be arranged so as to face each other. The terminal part 40 is provided at the one end 21A of the substrate 21-1. The terminal part 40 is electrically coupled to each of the three first optical sensors 10A via the first wiring line 26A.

The substrate 21-1 is provided with three first power supply electrodes 25A extending along the second direction Dy. Each of the three first power supply electrodes 25A is electrically coupled to the terminal part 40 of the substrate 21-1 via the first wiring line 26A and is supplied with the power supply signal from the power supply circuit 123 (refer to FIG. 3) via the terminal part 40. Each of the upper electrodes 15A of the three first optical sensors 10A is coupled to the first power supply electrode 25A via the conductor 24 and electrically coupled to the terminal part 40 via the first wiring line 26A coupled to the first power supply electrode 25A. With this configuration, the three upper electrodes 15A are supplied with power from independent power systems of the respective first power supply electrodes 25A.

As illustrated in FIG. 10, each of the three first optical sensors 10A includes the substrate 21-1, the photodiode PD, the third wiring lines 26C, and the insulating layer 27. The photodiode PD of the first optical sensor 10A includes the lower electrodes 11, the lower buffer layer 12, the active layer 13, the upper buffer layer 14, and the upper electrode 15 (15A).

Each of the three first power supply electrodes 25A is electrically coupled to the terminal part 40 of the substrate 21-1 via the first wiring line 26A, and is supplied with a power supply signal from the power supply circuit 123 via the terminal part 40. Each of the upper electrodes 15B of the three first optical sensors 10A is electrically coupled to a corresponding one of the first power supply electrodes 25A and electrically coupled to the terminal part 40 via the first wiring line 26A coupled to the first power supply electrode 25A. With this configuration, each of the three upper electrodes 15A is supplied with power from an independent system of a corresponding one of the first power supply electrodes 25A. Each of the lower electrodes 11 of the three first optical sensors 10A is electrically coupled to the terminal part 40 via the third wiring line 26C of the substrate 21-1.

The detection device 1 supplies a power supply signal from the power supply circuit 123 to the three upper electrodes 15A via the three first power supply electrodes 25A. In the detection device 1, the light source 60 is turned on to emit the light toward the finger Fg. The light source 60 emits the light toward one side and the other side in the circumferential direction 200C. In the detection device 1, the three first optical sensors 10A receive the light reflected by the finger Fg or the like. The detection device 1 detects the information on the living body of the finger Fg based on the amount of light detected by each of the two photodiodes PD of each of the three first optical sensors 10A.

Thus, by arranging the three first optical sensors 10A in the circumferential direction 200C of the housing 200, the detection device 1 can supply power from the three first power supply electrodes 25A to the upper electrodes 15A of the three first optical sensors 10A to operate the lower electrodes 11. Thus, even when the three first optical sensors 10A are arranged in the circumferential direction 200C of the housing 200, the detection device 1 can reduce differences in sensitivity among the three first optical sensors 10A by reducing differences in power supply capacity among the three upper electrodes 15A.

### Contrast with Reference Example Substrate According to Third Embodiment

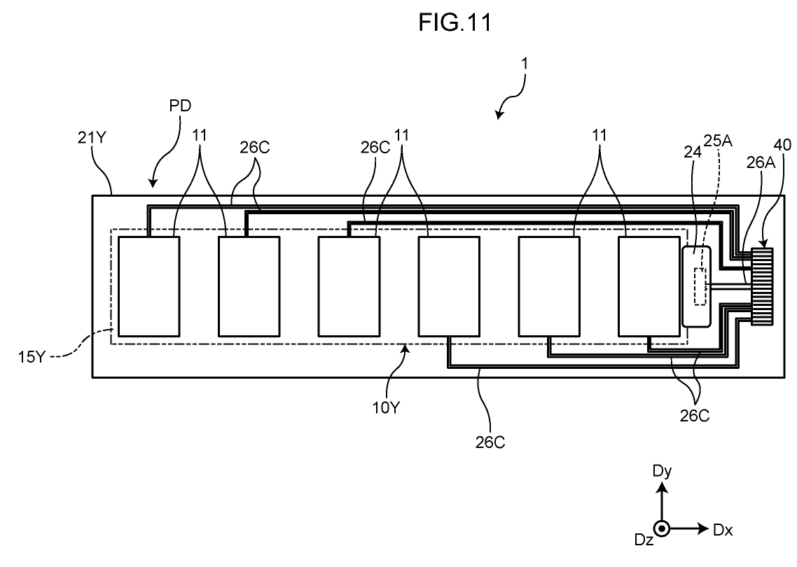

FIG. 11 is a schematic top view illustrating an exemplary configuration of a reference example substrate according to the third embodiment. On a reference example substrate 21Y illustrated in FIG. 11, six lower electrodes 11 are arranged along the first direction Dx, and one optical sensor 10Y is formed by disposing an upper electrode 15Y so as to cover all the six lower electrodes 11. The reference example substrate 21Y supplies power from one first power supply electrode 25A to the optical sensor 10Y. Thus, the six lower electrodes 11 on the reference example substrate 21Y are operated by supplying power to one upper electrode 15Y. Therefore, the resistance value of a path to the upper electrode 15Y is higher as the distance from the first power supply electrode 25A increases. Thus, a difference in power supply capacity occurs between an element farther from the first power supply electrode 25A and an element closer to the first power supply electrode 25A, resulting in a difference in sensitivity of elements in the optical sensor 10Y.

In contrast, the detection device 1 according to the third embodiment is configured to supply the power from the three first power supply electrodes 25A to the upper electrodes 15A of the three first optical sensors 10A. This configuration inhibits an increase in the resistance values of the paths to the three upper electrodes 15A in the detection device 1, so that the differences in sensitivity among the three first optical sensors 10A can be reduced. In addition, in the detection device 1 according to the third embodiment, even if a failure occurs in the elements of the three first optical sensors 10A, it is possible to prevent all the first optical sensors 10A from becoming unusable.

### Fourth Embodiment

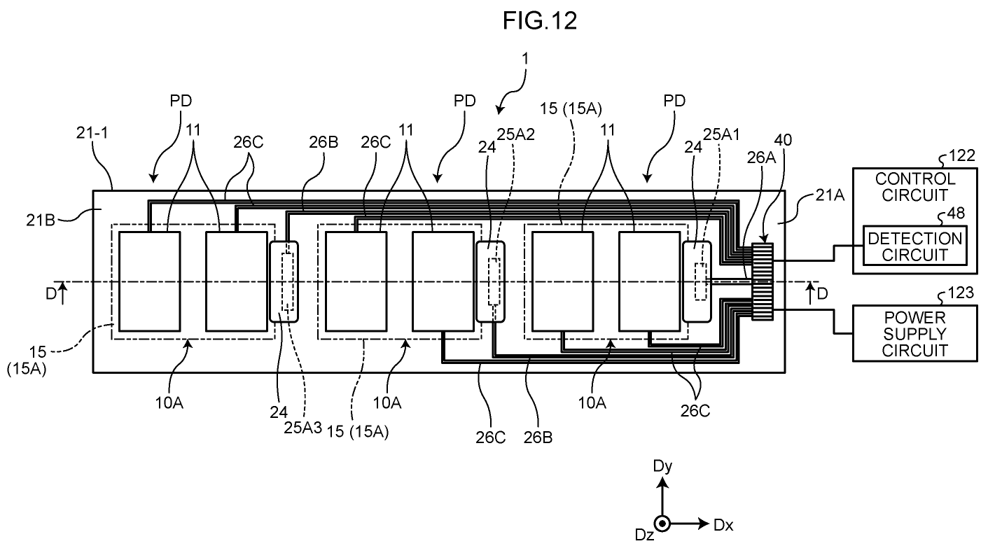

FIG. 12 is a schematic top view illustrating an exemplary configuration of the substrate 21-1 of the detection device 1 according to a fourth embodiment. In the fourth embodiment, the detection device 1 includes the substrate 21-1, the terminal part 40, and the three first optical sensors 10A described above. The substrate 21-1 includes a first power supply electrode 25A1, a first power supply electrode 25A2, and a first power supply electrode 25A3 that extend along the second direction Dy. The first power supply electrodes 25A1, 25A2, and 25A3 are formed so as to be larger in length in the second direction Dy of the substrate 21-1 as the distance from the terminal part 40 increases. The first power supply electrodes 25A1, 25A2, and 25A3 have the same length in the first direction Dx. That is, the electrode area of each of the first power supply electrodes 25A1, 25A2, and 25A3 increases with increasing distance from the terminal part 40. Each of the first power supply electrodes 25A1, 25A2, and 25A3 is electrically coupled to the terminal part 40 of the substrate 21 via the third wiring line 26C, and is supplied with the power supply signal from the power supply circuit 123 via the terminal part 40. Each of the first power supply electrodes 25A1, 25A2, and 25A3 is electrically coupled to the upper electrode 15A of a corresponding one of the first optical sensors 10A via the conductor 24 and electrically coupled to the terminal part 40 via the second wiring line 26B. As a result, each of the upper electrodes 15A of the three first optical sensors 10A is supplied with power from independent systems of the first power supply electrodes 25A1, 25A2, and 25A3.

Thus, even when the distance from the terminal part 40 to each of the first power supply electrodes 25A1, 25A2, and 25A3 sequentially increases on the substrate 21-1, the detection device 1 can supply substantially the same power from the first power supply electrodes 25A1, 25A2, and 25A3 to the upper electrodes 15A. As a result, in the detection device 1, the difference in sensor sensitivity can be reduced even when the distances from the first optical sensors 10A to the terminal part 40 differ in the circumferential direction 200C of the housing 200.

In each of the embodiments described above, the case has been described where the detection device 1 accommodates the substrate 21 or 21-1 and so forth in the ring-shaped housing 200, but the present disclosure is not limited to this case. The detection device 1 may, for example, be accommodated in a rectangular housing, or be configured to be attached to the object to be measured without being accommodated in a housing.

In the embodiments of the detection device 1, the case has been described where the wiring lines are coupled to the first power supply electrode 25A, the second power supply electrode 25B, the second power supply electrode 25C, and the like, but the detection device 1 is not limited to this case. For example, the detection device 1 may use the distal ends of the wiring lines as the first power supply electrode 25A, the second power supply electrode 25B, the second power supply electrode 25C, and the like.

The components in the embodiments described above can be combined as appropriate. Other operational advantages accruing from the aspects described in the embodiments of the present disclosure that are obvious from the description herein, or that are conceivable as appropriate by those skilled in the art will naturally be understood as accruing from the present disclosure.

What is claimed is:

1. A detection device comprising:
a substrate having opposite ends in a first direction with a notch therebetween;
a terminal part provided at one end in the first direction of the substrate;
a first optical sensor provided on the substrate between the notch and the terminal part; and
a second optical sensor provided on the substrate between the notch and another end of the substrate, wherein
in each of the first optical sensor and the second optical sensor, a lower electrode, a lower buffer layer, an active layer, an upper buffer layer, an upper electrode, and a sealing film are stacked on the substrate in the order as listed,
the upper electrode of the first optical sensor is coupled to a first power supply electrode and coupled to the terminal part via a first wiring line coupled to the first power supply electrode,
the upper electrode of the second optical sensor is coupled to a second power supply electrode and coupled to the terminal part via a second wiring line coupled to the second power supply electrode, and
the lower electrodes of the first optical sensor and the second optical sensor are coupled to the terminal part via third wiring lines.

2. The detection device according to claim 1, wherein the first wiring line and the second wiring line are metal lines in the same layer as the third wiring lines coupled to the lower electrodes on the substrate.

3. The detection device according to claim 2, wherein
the first power supply electrode is provided between the one end side of the substrate and the first optical sensor in the first direction, and
the second power supply electrode is provided between the other end side of the substrate and the second optical sensor in the first direction.

4. The detection device according to claim 3, further comprising a light source located in the notch of the substrate.

5. The detection device according to claim 4, wherein the second power supply electrode is formed to be longer in length in a second direction intersecting the first direction than the first power supply electrode.

6. The detection device according to claim 4, wherein the terminal part is coupled to a flexible printed circuit board.

7. The detection device according to claim 6, wherein ends of the sealing films of the first optical sensor and the second optical sensor and the first power supply electrode are integrally covered with a resin.

8. A detection device comprising:
a substrate extending in a first direction;
a terminal part provided at one end in the first direction of the substrate;
a plurality of optical sensors arranged along the first direction on the substrate, wherein
in each of the optical sensors, a lower electrode, a lower buffer layer, an active layer, an upper buffer layer, an upper electrode, and a sealing film are stacked on the substrate in the order as listed,
each of the upper electrodes of the optical sensors is coupled to a power supply electrode and coupled to the terminal part via a first wiring line coupled to the power supply electrode, and
each of the lower electrodes of the optical sensors is coupled to the terminal part via a third wiring line.

9. The detection device according to claim 8, wherein a plurality of the power supply electrodes provided on the substrate become larger in length in a second direction intersecting the first direction on the substrate with increasing distance from the terminal part.
